(19) United States
(12) Patent Application Publication
(10) Pub. No.: US 2020/0261302 A1
(43) Pub. Date: Aug. 20, 2020

(54) iCane The Intelligent Cane: Alternative to tactile paving for the blind

(71) Applicant: Neil Mitra, WATERLOO (CA)

(72) Inventor: Neil Mitra, WATERLOO (CA)

(21) Appl. No.: 16/278,503

(22) Filed: Feb. 18, 2019

(51) Int. Cl. A61H 3/06; H04W 4/029; A61B 5/00

(52) U.S. Cl. CPC A61H 3/068; A61H 3/061; A61H 2201/5092; H04W 4/029; A61B 5/0022; A61H 3/066

(57) ABSTRACT

The robotic cane will follow the markings on roads and pavements with hunter green and black colored thermoplastic paint, thereby ensuring the mobility of blind people. It will enable to monitor heath conditions of the individuals, track the locations of the individuals, alert individuals about obstructions along the pathways, and has a voice-activated app to control the functionalities of the cane. This will eliminate the use of expensive tactile paving, thereby, providing simple and cost-effective solution for blind people to navigate both indoor and outdoor environments.

### CROSS REFERENCE TO RELATED APPLICATIONS

This application claims priority to U.S. provisional patent application No. 62/485,339 filed on Apr. 13, 2017^{[5]}, the entire contents of this is incorporated by reference herein.

### FIELD OF INVENTION

The present invention relates generally to find a solution that includes the use of a robotic cane which can follow a thermoplastic paving in order for the blind person to walk more freely without dangers, have ability to recognize an obstacle and maneuver around it, ability to monitor health condition of the individual and also APP enable location finder for critical infrastructures and ability to be traced by others. This will also involve finding out which thermoplastic paint color is best to use for such robotic iCane to follow more meticulously the roads and pavements (side-walks) with minimal error.

### BACKGROUND OF THE INVENTION

A system of textured surface on the ground found mainly on sidewalks, stairs, and train station platforms are called tactile paving. They are mainly yellow in color. Tactile paving is used to navigate the blind and visually impaired pedestrians from one place to another. There are many problems with this system, such as the blind person must know where the paving is in order to follow it and also the noise of the cane needs to be audible clearly which might become a problem with the surrounding noise. That is not only the problem but there is a high cost of tactile paving associated with it.

For example in the city of Toronto, Canada, where there are a few tactile paving, the paved area of Toronto is 620,000 m2^{[2]} and if this area were to be tactile paved the total cost would be USD$6,302,000^{[3]} (with the cheapest tactile paving which costs USD$10 per m2) and USD$31,570,000 (with the highest quality tactile paving which costs USD$50 per m2).

Hence, if one uses iCane that can follow a black thermoplastic paint line, the cost of thermoplastic paint through the whole city of Toronto would be USD$167,550. In total if we subtract the cost of the cheap tactile paving from our system there is a USD$6,144,450 save for the government and if we subtract the premium tactile paving from our system there is a USD$31,352,450 save for the government.

The robotic iCane uses eight Infrared sensors connected to Arduino Uno. It also includes an ultrasonic sensor which detects obstacles so that the iCane automatically guides the blind person away from the obstruction by maneuvering itself from the obstruction. There is also medical devices such as heart rate sensor is added, which displays the pulse reading of the visually impaired individual in case if the individual needs medical attention or undergoing some trauma. An android app system is also added to the iCane which will allow the visually impaired individual to be guided to their destination. The iCane app will also show nearby hospitals, restaurants, and schools via voice activation. The iCane will display the current location of the blind individual so their respective family members can track their location and whereabouts, Due to the use of advanced infrared sensors the width of the thermoplastic paint track can be reduced. The robots use sensors like cameras, to observe the world around them and know how to react—just like our eyes (or a visually impaired person would use a cane and their sense of touch) to observe a trail or path in front of you. The robot relies on a sensor that detects infrared (IR) light (which is part of the electromagnetic spectrum, just outside the range of visible light that humans can see). The sensor consists of two parts: an IR emitter which sends out IR light, and an IR detector, which detects incoming IR light. When combined, these two parts can be used to detect a nearby bright object, like white paper. The IR emitter sends out IR light, which bounces back off the white paper, and is “seen” by the IR detector. If the surface is too dark, the IR light will be absorbed by the surface instead of bouncing back from it, so the IR detector will not “see” any IR light. This would help the blind person to commute freely from one place to another as the robotic iCane would follow the black line on any surface.

Here, a light colored (white) board was used where different colored track were painted to see which color allowed the robotic iCane most accurately to follow the track. The result observed was color black. This can be applied in real life situations as the sidewalks are colored grey, however the sensors sense grey as the color white causing the robotic iCane to move.

Assemble the robot chassis that consists of one brown plastic flat base, wheels, and two DC motors with screws.

Solder the pinheads to the Infrared Sensor and the Motor Driver for connections.

To calibrate the sensors connect the sensor pins to the Arduino digital ports

Assemble the circuits by connecting the IR sensor and Arduino Uno.
The sensor pins 0-7 to digital pins 3-10 respectively
The sensor VCC and GND to Arduino 5V and GND
The sensor LED to Arduino pin 2
Upload the code from the Polulo library^{[7]} to the Arduino

Calibrate the IR sensor by facing down the IR sensor on a blank white piece of paper with black electrical tape in the middle. Within ten seconds move the sensor across the surface slowly allowing each sensor to read the white and black surface showing the results in the serial monitor in the Arduino IDE software, where 1000 being the darkest (black) and 0 being the lightest (white).

Mount the IR sensors on the robot by using nails which fit through the front holes in the chassis. Use a Popsicle stick to attach the sensor to the nails by hot gluing them together. The sensor is placed in such a way that it is 3 mm off the ground for the robot to work properly.

For making the track following robot, take off the connections of the calibration phase. Place the motor driver on top of the Arduino and secure it onto the chassis by using double-sided foam tape. Make the following connections from the infrared sensors to the motor driver:
The Sensor VCC connect to the Arduino +5V
The Sensor GND connect to the Arduino Ground
The Sensor pin 2 connect to the Arduino Analog 0
The Sensor pin 3 connect to the Arduino Analog 1
The Sensor pin 4 connect to the Arduino Analog 2
The Sensor pin 5 connect to the Arduino Analog 3
The Sensor pin 6 connect to the Arduino Analog 4

Connect the Left Motor to the M1 jack and the right motor to M2 jack on the motor driver. Hot glue a wire to one end of a switch and attach it to the +ve M jack of the motor driver. Attach a battery buckle to a 9 v battery, then take the positive end of the battery and hot glue it to the other side of the switch. The negative output from the battery attaches to the GND of the motor driver. Then code the Arduino^{[1]}.

Next step is to simulate the alternate to tactile paving on paper. The alternate to tactile paving involves painting guide lines of different colors on side walk which will allow the iCane to follow the desired pathway. Hence we have performed 4 sets of experiment which includes:
a. Different line thickness (5 mm, 15 mm, 35 mm, 55 mm, and 75 mm) in order to find which track width would be the most efficient for the iCane to follow.
b. Black colored track for the iCane robot to follow on different color backgrounds, simulating black colored guide path on different colored sidewalk.
c. Different colored (black, yellow, blue, red, and hunter green) straight line tracks for the iCane robot to follow on white background, simulating different colored guide paths on natural colored sidewalks.
d. Different colored (black, yellow, blue, red, and hunter green) curved tracks for the iCane robot to follow on white background, simulating turns on different colored guide paths on natural colored sidewalks.

For making the ultrasonic sensor secure an Arduino Uno and a breadboard using double-sided tape to the chassis. Make the following connections for the ultrasonic sensor to the Arduino:
HC-SR04 sensor attach to the Breadboard
Sensor VCC connect to the Arduino Board +5V
Sensor GND connect to the Arduino Board GND
Sensor Trig connect to the Arduino Board Digital I/O 9
Sensor Echo connect to the Arduino Board Digital I/O 10

Then connect the breadboard, buzzer, and LED:
Buzzer attach to the Breadboard
Buzzer long leg (+ve) connect to the Arduino Board Digital 11
Buzzer short leg (−ve) connect to the Arduino Board GND
LED attach to the Breadboard
Resistor connect to the LED long leg (+)
Resistor other leg (from LED's long leg) connect to the Arduino

Board Digital 13
LED short leg (−) connect to the Arduino Board GND

Next step performed was to test the ultrasonic sensor for which make rectangular prisms of different heights ranging from (1″, 2″, 3″, and 4″) and see if the ultrasonic sensor detects it or not. Then make different shapes (triangle, sphere, and cylinder) with the same height that the ultrasonic sensor detected to see whether the shapes have an effect or not.

Then to make the Heart Pulse Rate Sensor the following connections was done with the Arduino Uno, Potentiometer, and LCD

### Pulse Rate Sensor Connections:

(−) connects to Ground.
(+) connects to 5 v.
Out pin connects to Arduino Analog 0

### LCD Connections:

VSS to Ground.
VDD to 5 v.
VO to 10 k Potentiometer
RS to Arduino Digital 12
RW to Ground.
E to Arduino Digital 11
D4 to Arduino Digital 5
D5 to Arduino Digital 4
D6 to Arduino Digital 3
D7 to Arduino Digital 2
A to 5 v.
K to Ground.

Then testing of the response time taken for the Heart Pulse Rate Sensor was conducted to find out how long it takes for the sensor to get the correct values.

Then to verify whether the Pulse Heart Rate Sensor works, plug in the Arduino (with the code and setup running) into your computer and run the serial monitor and you can see your heart rate (bpm). This proves that your Pulse Heart Rate sensor is working.

Next step performed was coding for the GPS navigation through Android Application.

Then testing for the response time of the App was determined to find the efficiency of the app.

The focus of the present invention to find a way that can eliminate the use of expensive tactile paving and find an alternative cost-effective solutions for blind people to navigate roads. The iCane contains an app which will allow nearby family members to locate their respective visually impaired individual as well as allowing the blind person to navigate key utilities such as hospitals, schools and restaurants. Further additions on the iCane are an ultrasonic sensor alarm, which will allow blind person to know any obstacles on the track and would maneuver the obstacle and also the heart rate sensor, which will monitor the health condition in case of any medical emergency.

### SUMMARY OF THE INVENTION

One aspect of the invention provides a iCane with robot containing 8 infrared (IR) sensors able to track defined pathways.

A further aspect of the invention provides the iCane to follow any darker color pathways that infrared light can detect.

A further aspect of the invention provides the iCane to assist the visually impaired individuals.

A further aspect of the invention provides the iCane to follow a track width of 15 mm.

A further aspect of the invention provides the iCane to detect any kind of obstructions that is 3 inches or more.

A further aspect of the invention provides the iCane to monitor the heart pulse rate.

A further aspect of the invention provides the iCane to follow a voice activated app.

### BRIEF DESCRIPTION OF THE DRAWINGS

The forgoing and other aspects of the invention will become more apparent from the following description of specific embodiments thereof and the accompanying drawings which illustrate, by way of example only, the principle of invention. In the drawings:

FIG. 1 shows the apparatus with the cane attached to the robot chasis.

FIGS. 2 and 2a shows how the different parts of the robots are attached to the chasis of the robot. The details name of the parts are also mentioned.

FIG. 3 shows how the robot is being calibrated and sensors attached to the chasis.

FIG. 4 shows how the robot follows the different color straight tracks and which color works best.

FIG. 5 shows how the robot follows the different color curved tracks and which color works best.

FIG. 6 shows how the robot follows the black straight line in different color pathway.

FIG. 7 shows how much time taken for the robot to travel different millimeter black track on a white background. It was observed that the robot took least time on 15 mm track width, deviated off the track on 5 mm, and did not move at all on 75 mm track width.

FIG. 8 shows the observation that was done with straight tracks colored blue, yellow, green, red, and black and measure the amount of deviation of the wheel of robot in centimeters. It was observed that the robot went straight on black and green color whereas in the other colors the wheel deviated right. This shows that both black and green color can be used as a thermoplastic paint on street sidewalks. The experiment was repeated 5 times and the table above shows the average results that was observed.

FIG. 9 shows how much time taken for the robot to travel on the 15 mm thick black line on non-white backgrounds i.e., blue, yellow, red, and green. It was observed that the robot took the most time to travel along the black line with hunter green background and least time with red and yellow background. This suggests that the IR sensors on the robot can only work on light background. Therefore the thermoplastic paint would only work if the background pavement color is white or any such lighter shades.

The same experiment was done using curved tracks with different colors. Here also the robot followed the black and green color tracks. Hence this conclusively suggests that the replacement of the tactile pavement should be done with black or green color thermoplastic paint on street sidewalks.

FIG. 10 (a) shows the observation that was done with the ultrasonic sensor on rectangular obstacles with different heights in inches It was observed that obstacle of height 3 inches and more could be detected by the ultrasonic sensor.

FIG. 10 (b) shows the observation that was done with different shapes of obstacles with 3 inches height to see which shapes works. It was observed that shapes have no effect on the sensor.

FIGS. 11(a) and 11(b) shows the observation time for pulse heart rate sensor and the graph of pulse rate mapped on Arduino serial monitor. The experiment was done five times and it was observed that the average response time taken was 9.958 seconds.

FIG. 12 shows the observation for the response time of the APP for the different locations/places. The experiment was done five times. It was observed that average time taken for the hospital was 2.39 sec., for school it was 2.40 sec., and for restaurant it was 2.42 seconds.

### DETAILED DESCRIPTION OF AN EMBODIMENT

The description which follows, and the embodiments describe therein, are provided by way of illustration of an example, or examples, of particular embodiments of the principles of the present invention. These examples are provided for the purpose of explanation, and not limitation, of those principles and of the invention.

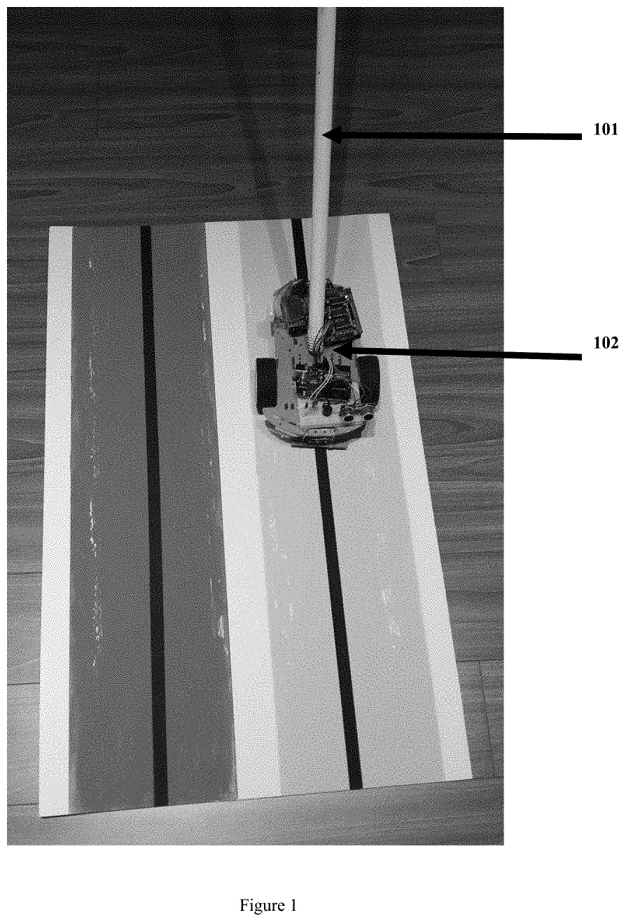

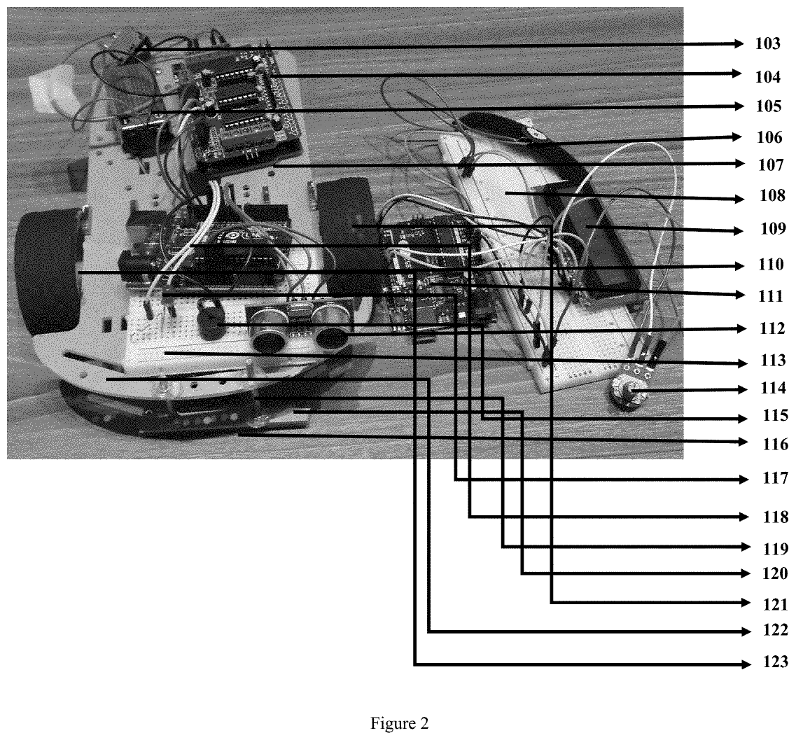

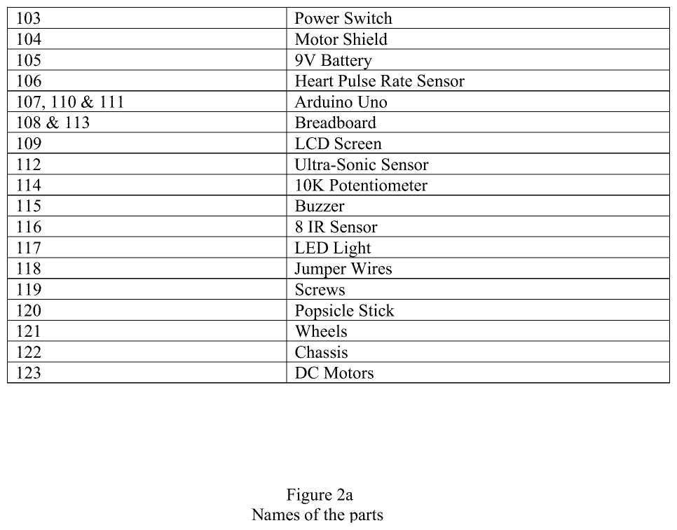

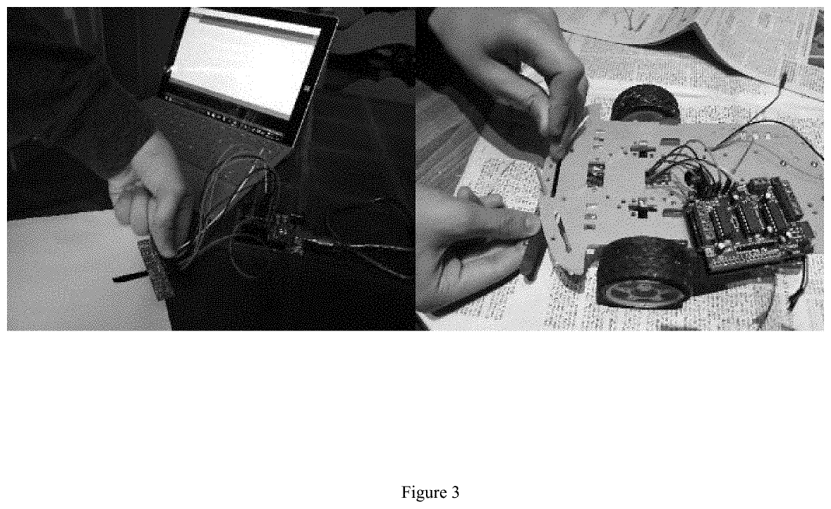

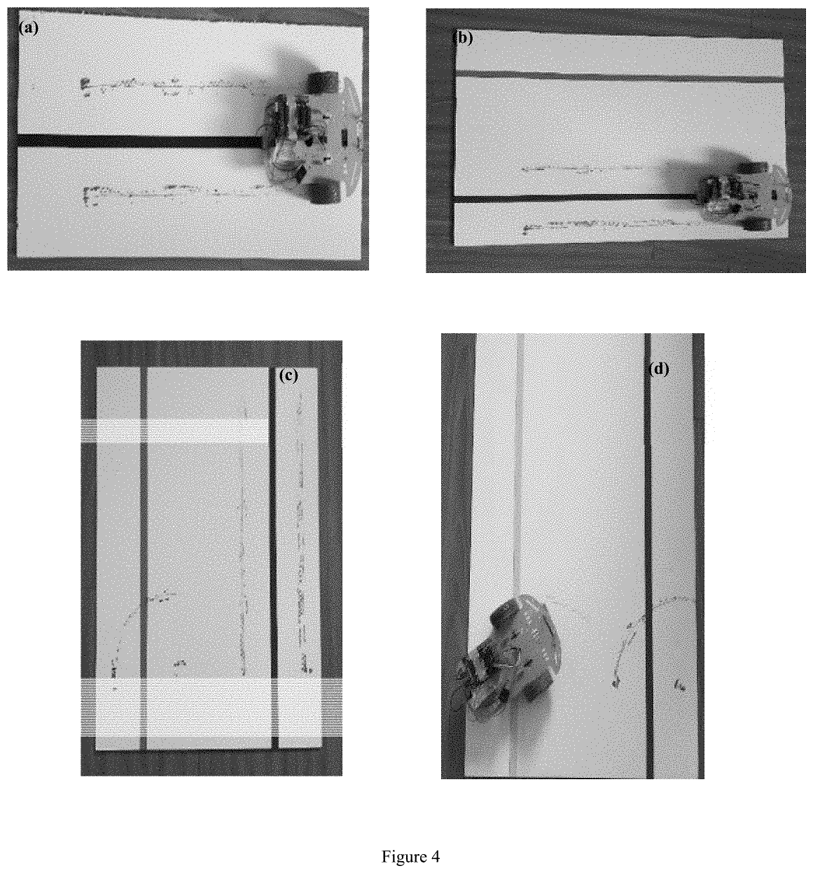

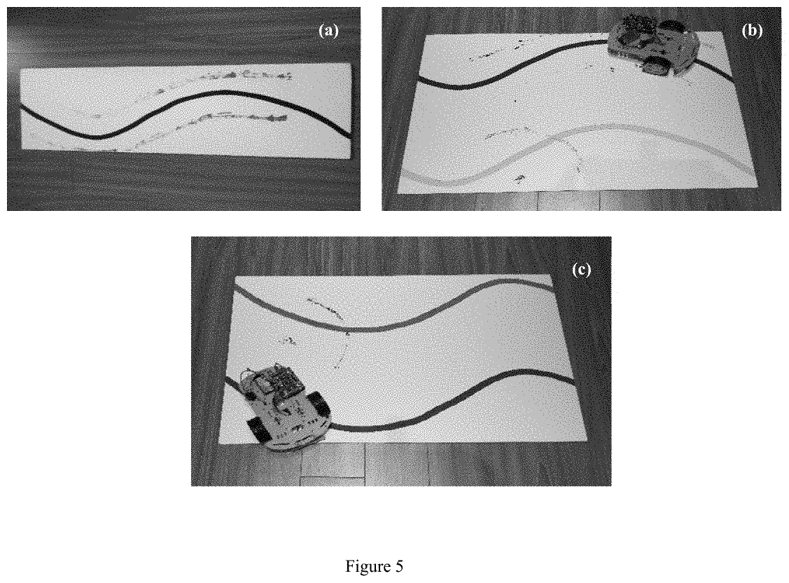

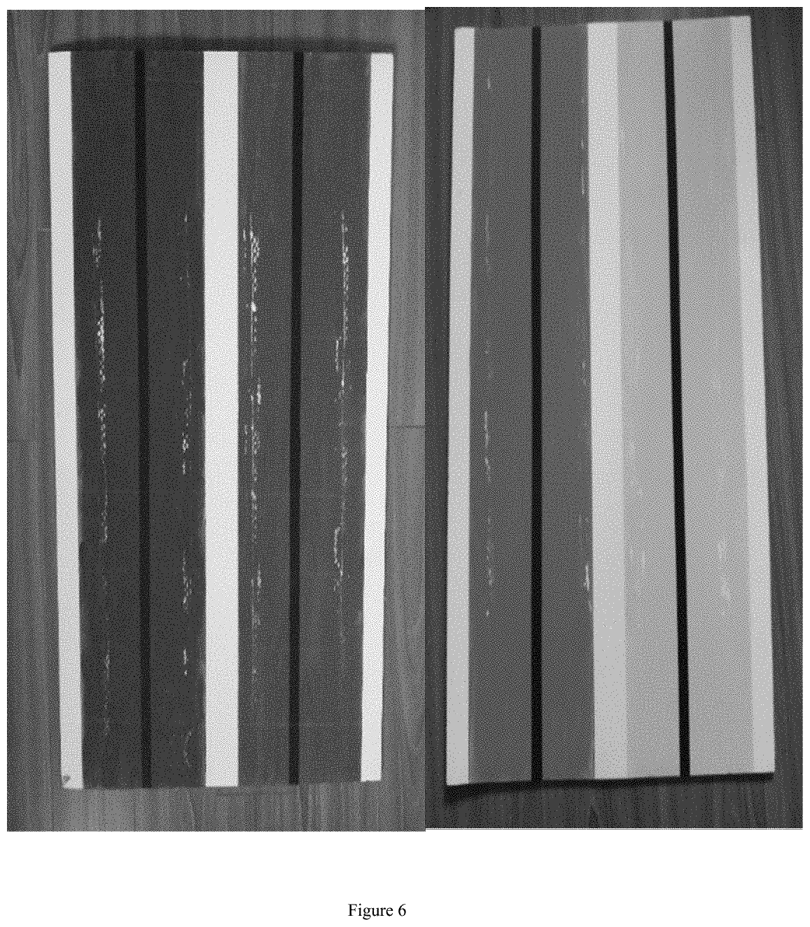

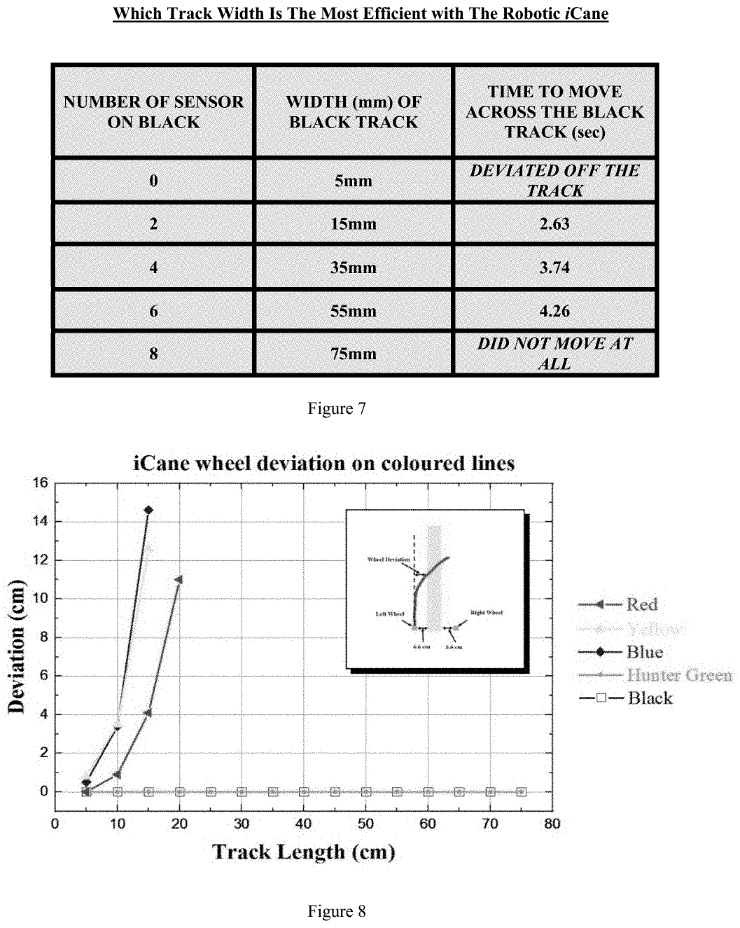

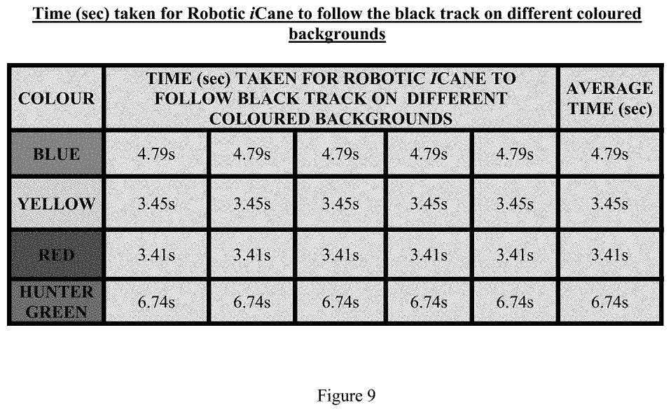

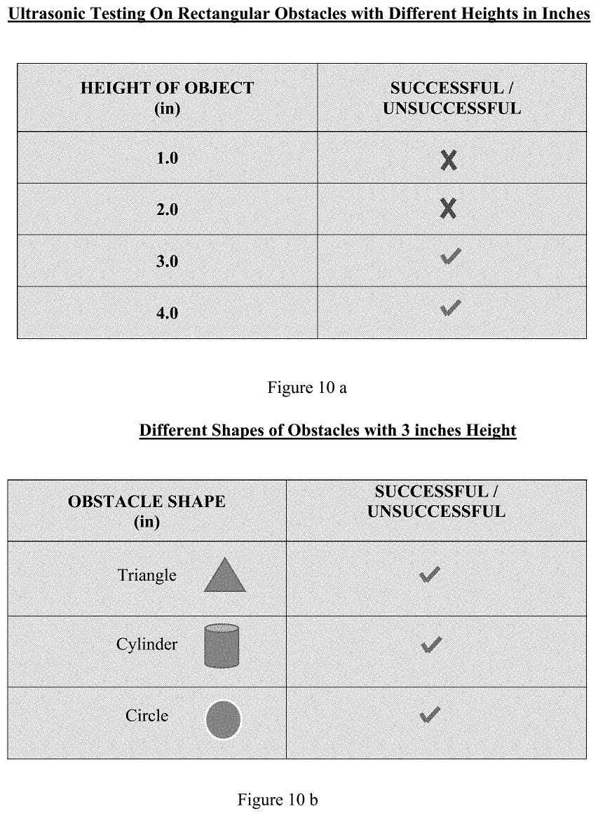

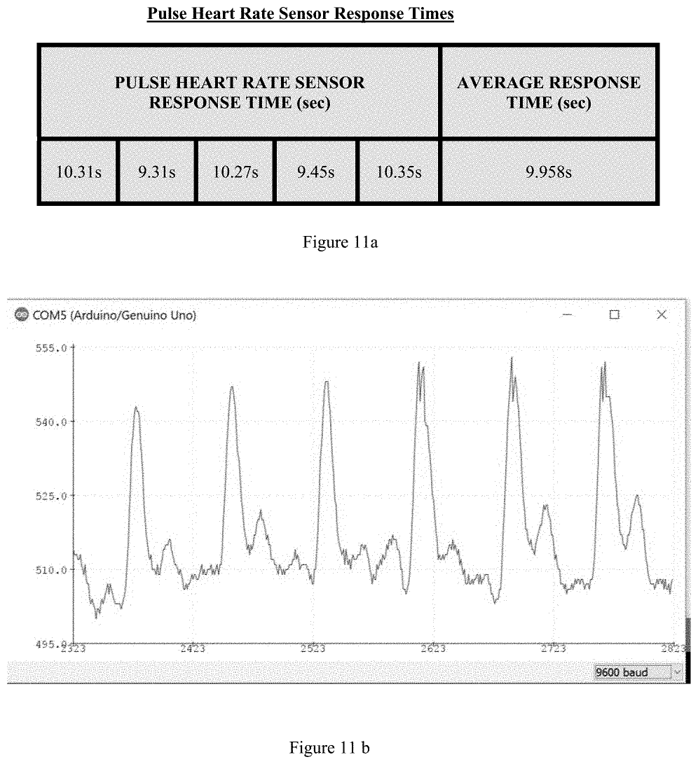

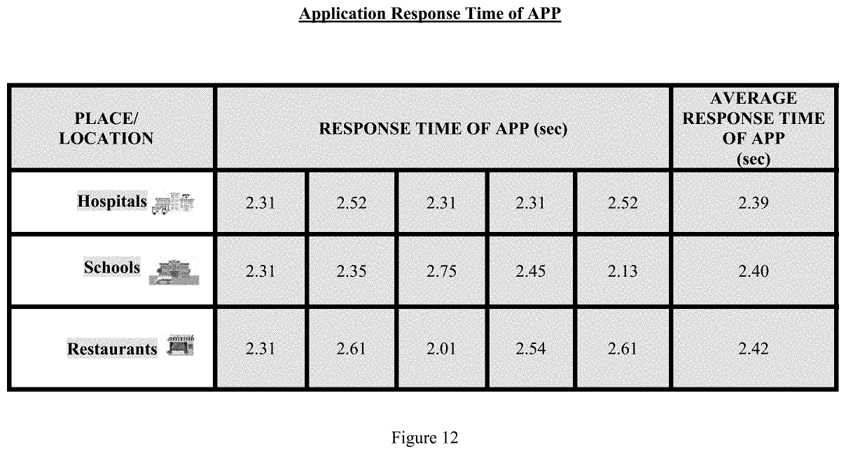

The testing apparatus of the invention is shown in FIG. 1. As shown in FIG. 1, the testing apparatus comprises of a cane 101, and a robot 102.

The robot 102 consists of the different parts. The robot has 2 DC motors 123 attached between the chassis 122 and wheels 121 on both sides. The robot consists of power switch 103; jumper wire 118; breadboard 108 & 113; Arduino Uno 107, 110 & 111; Motor Shield 104; 9 V battery 105; heart pulse rate sensor 106; LCD screen 109; ultrasonic sensor 112; 10 K potentiometer 114; buzzer 115; 8 infrared (IR) sensor 116; LED light 117; Screws 119; Popsicle stick 120.

The cane 101 is attached to the robot 102 with screw.

The robot chassis 122, motors 123 and wheels 121 are assembled. Then solder the pinheads to the IR sensor and the motor shield 104.

Calibration of the IR sensors 116 were done by connecting the sensor pins to the Arduino Uno 107 digital ports and uploading the code from the Polulo library [4] to the Arduino:
The sensor pins 0-7 to digital pins 3-10 respectively
The sensor VCC and GND to Arduino 5V and GND
The sensor LED to Arduino pin 2
   Then the IR sensors were placed facing down on a blank white piece of paper with black electrical tape in the middle. Within ten seconds move the sensor across the surface slowly allowing each sensor to read the white and black surface showing the results in the serial monitor in the Arduino IDE software, where 1000 being the darkest (black) and 0 being the lightest (white).

Then mount the IR sensors 116 on the robot chassis 122 by using screws 119 which fit through the front holes in the chassis. Then Popsicle stick 120 was used to attach the sensor to the nails by hot gluing them together. The sensor were placed about 3 mm off the ground for the robot to work properly.

To make the track following robot, place the motor driver 104 on top of the Arduino Uno 107 and secure it onto the chassis by using double-sided foam tape. Then the following connections from the infrared sensors to the motor driver was done:
The Sensor VCC connect to the Arduino +5V
The Sensor GND connect to the Arduino Ground
The Sensor pin 2 connect to the Arduino Analog 0
The Sensor pin 3 connect to the Arduino Analog 1
The Sensor pin 4 connect to the Arduino Analog 2
The Sensor pin 5 connect to the Arduino Analog 3
The Sensor pin 6 connect to the Arduino Analog 4

Then connect the Left Motor 123 to the M1 jack and the right motor 123 to M2 jack on the motor driver 104. Hot glue a wire to one end of a switch 103 and attach it to the +M jack of the motor driver 104. Attach a battery buckle to a 9 v battery 105, then take the positive end of the battery and hot glue it to the other side of the switch. The negative output from the battery 105 attaches to the GND of the motor driver 104.

Then the next step was to simulate the alternate to tactile paving on paper. The alternate to tactile paving involves painting guide line of different colors on sidewalk which will allow iCane to follow the desired pathway. Hence 4 sets of experiments were performed which includes:
(a) Black colored track for the iCane robot to follow on different color backgrounds, simulating black colored guide path on different colored sidewalk.
(b) Different colored (black, yellow, blue, red, and hunter green) straight line tracks for the iCane robot to follow on white background, simulating different colored guide paths on natural colored sidewalks.
(c) Different colored (black, yellow, blue, red, and hunter green) curved tracks for the iCane robot to follow on white background, simulating turns on different colored guide paths on natural colored sidewalks.
(d) Different line thickness (5 mm, 15 mm, 35 mm, 55 mm, and 75 mm) in order to find which track width would be the most efficient for the iCane to follow.

Then to make the ultrasonic sensor 112 secure an Arduino Uno 110 and a breadboard 113 using double-sided tape to the chassis 122. Following connections for the ultrasonic sensor to the Arduino Uno was done:
HC-SR04 sensor attach to the Breadboard
Sensor VCC connect to the Arduino Board +5V
Sensor GND connect to the Arduino Board GND
Sensor Trig connect to the Arduino Board Digital I/O 9
Sensor Echo connect to the Arduino Board Digital I/O 10

The following connections were done for the breadboard 113, buzzer 115, and LED light 117:
Buzzer attach to the Breadboard
Buzzer long leg (+ve) connect to the Arduino Board Digital 11
Buzzer short leg (−ve) connect to the Arduino Board GND
LED attach to the Breadboard
Resistor connect to the LED long leg (+)
Resistor other leg (from LED's long leg) connect to the Arduino Board Digital 13
LED short leg (−) connect to the Arduino Board GND

Next step performed was the testing of the ultrasonic sensor for which rectangular prisms of different heights ranging from (1″, 2″, 3″, and 4″) were made to see if the ultrasonic sensor detects it or not.

Then different shapes (triangle, sphere, and cylinder) were made with the same height that the ultrasonic sensor detected to see whether the shapes have an effect or not.

Then the Heart Pulse Rate Sensor 106 were made by doing the following connections with the Arduino Uno 111, 10 K Potentiometer 114, and LCD 109. Following connections with the Pulse Rate Sensor and Arduino Uno was done:
(−) connects to Ground.
(+) connects to 5 v.
Out pin connects to Arduino Analog 0

Then the following connection of the LCD, potentiometer and Arduino Uno were done:
VSS to Ground.
VDD to 5 v.
VO to 10 k Potentiometer
RS to Arduino Digital 12
RW to Ground.
E to Arduino Digital 11
D4 to Arduino Digital 5
D5 to Arduino Digital 4
D6 to Arduino Digital 3
D7 to Arduino Digital 2
A to 5 v.
K to Ground
Code the Arduino^{[6]}

Then testing of the response time taken for the Heart Pulse Rate Sensor was done to find out how long it took for the sensor to get the correct values.

To verify if the Pulse Heart Rate Sensor works, plug in the Arduino (with the code and setup running) into the computer and run the Arduino serial monitor and the heart rate (bpm) graph can be seen. This proves that the Pulse Heart Rate sensor is working.

Next step performed was coding the GPS navigation through Android Application.

Then testing of the response time of the App was done to determine the efficiency of the app.

The working principle of the apparatus generally involves in switching ON the power switch and moving the IR sensor over the black track from left to right and holding the cane on its path. The robot will guide the person automatically along the path and will stop or move away when there is a hazard.

### EXAMPLE

The following specific example demonstrates one embodiment of the present invention, and is provided for the purposes of explanation, and not limitation, of the present invention.

Wherein the parts of the robot were obtained from Amazon India, Arduino Uno from Arduino store, motor shield from Banggood.com, Polulu QTRC 8RC Reflectant IR sensor from Polulu store, 10K potentiometer, ultrasonic senor, buzzer and Heart Pulse rate sensor from Amazon Canada. The batteries were obtained from Energizer. The cane, Popsicle sticks, and double sided tape were obtained from Walmart, Canada.

### REFERENCES

1. https://www.youtube.com/watch?v=wbrt2ClgZik&t=247s for the method to make infrared line follower and code Arduino, accessed on Nov. 12, 2017
2. https://www.google.ca/search?q=total+paved+area+of+Toronto&oq=total+paved+area+of+Toronto&aqs=chrome..69i57.7949j0j8& sourceid=chrome&ie=UTF-8#q=area+of+Toronto, accessed on Dec. 28, 2016.
3. https://www.alibaba.com/product-detail/Bluestone-pavers-blind-tiles-tactile-paving_60586996670.html?spm=a2700.7724838.0.0.9EdWTa&s=p, accessed on Dec. 28, 2016.
4. https://www.alibaba.com/product-detail/Tactile-Paving-Blind-Stone-Pedestrian-Tactile_60239426431.html, accessed on Dec. 28, 2016.
5. N. Mitra, “iCane The Intelligent Cane: Alternative to tactile paving for the blind”, U.S. Provisional Patent No. 62/485,339, filed on Apr. 13, 2017.
6. https://www.hackster.io/vandenbrande/arduino-simple-heart-beat-monitor-with-lcd1602a-db7ba0, accessed on Jan. 27, 2018
7. https://www.pololu.com/docs/0J19/all, accessed on Nov. 16, 2017

We claim:

1. iCane which is a successful alternative to tactile paving for the blind by using the invention to follow a thermoplastic paint line decreasing the cost to implement and increasing efficiency mobility for blind individuals.

2. The Application of ultrasonic sensors and proximity sensors in an invention which detects obstructions on any thermoplastic paint line and avoids the obstruction and regain its initial path following the thermoplastic paint line.

3. An App will wirelessly send the GPS co-ordinates to the invention which will respectively assign a route of thermoplastic paint by using infrared sensors so that the invention can follow the track to the respective destination helping blind individuals.

4. The iCane of claim 1, wherein with robot containing 8 infrared sensor able to track defined pathways.

5. The iCane of claim 1, wherein the apparatus consists of:
(a) Brown plastic robot chassis (includes wheels, bovine wheel, and DC motors); (b) 9V batteries (×3); Battery Buckle (×3); (c) Breadboard (×2); Power Switch; (d) Jumper Wires (all male to male, female to female, and male to female); (e) QRT-8RC Reflectance/IR sensors (8); (f) Arduino Uno (×3); (g) Board Motor Shield L293D; (h) 25-pin 0.1″ header strip; (i) Ultrasonic Sensor HC-SR0F; (j) LED, Buzzer; (k) 220 ohm resistor (red, red, brown, gold stripes); (l) 10 k Potentiometer; (m) Pulse Heart Rate Sensor SEN-11574; (n) LCD Screen; (o) Arduino IDE program; (p) Android Studio version 2.3.0; Android phone (any company); Android drivers (needs computer); (q) 5 different colors: Red, Black, Yellow; Green, Blue; (r) Small Phillips-head screwdriver; Double-sided foam tape; Hot glue gun; Soldering iron and silver bearing flux solder; (s) Flat, open space to make a line-following track; (t) Needle-nose pillars or tweezers (make it easier to handle small circuit components); (u) Long Screwdriver (2), half of popsicle stick (used to attach the IR sensors to the front of the robot); (v) White poster boards (at least 4 pieces that you can arrange in a 2×2 grid on the floor). If you have more floor space, you can make a larger track with a 3×3 or even a 4×4 grid.

6. The apparatus of claim 1, wherein the invention can follow any color pathways.

7. The apparatus of claim 1, wherein is an assisting device for the visually impaired individuals.

8. The iCane of claim 1, wherein there is an application of thermoplastic paint for blind people to follow using any invention.

9. The iCane of claim 1, wherein thermoplastic paint can be used in any darker range infrared light for the invention to follow the line and help the blind person.

10. The App of claim 3, wherein an App allows blind individual's family members to know their current location.

11. The App of claim 3, wherein an App which shows the direction to nearby locations or any location for blind people to follow, via voice activation.

12. The iCane of claim 1, wherein the invention's voice activation will allow the invention to come to the user within a 100 meter radius of the invention by using built-in microphones which can detect where the user's voice is coming from, and then the invention will be able to maneuver around carefully around the proximity by using the ultrasonic sensor for obstacle detection.

13. The apparatus of claim 1, wherein an application of pulse heart rate sensor which will be able to show the users heart rate, SpO₂ (estimation of arterial oxygen saturation) level, and O₂ (oxygen) levels in the invention which follows a thermoplastic paint line for the blind.

14. The apparatus of claim 1, wherein an application of vital readings to be sent to a hospital database as well as the family members being able to know that there has been a medical emergency on the user via the invention's app on the invention capable of following a thermoplastic paint line for the blind.

15. The apparatus of claim 1, wherein an application of voice control on the invention to help visually impaired individuals.

16. The apparatus of claim 1, wherein an application of speed control on the invention's cane to increase speed or decrease speed of the robot which is following the thermoplastic line therefore allowing the blind individual to physically exercise.

17. The apparatus of claim 1, wherein an application of button on the invention's cane to activate voice activation to follow black thermoplastic paint line.

18. The apparatus of claim 1, wherein an ergonomic design of the placement of the controls of the invention's cane.

19. The apparatus of claim 1, wherein a Compact design of the invention and application of motherboard chips for an invention that can follow a thermoplastic paint line for the blind.

20. The application of claim 2, wherein an application of using any number of infrared sensors in the invention to follow a thermoplastic paint line for the blind with using any number of ultrasonic sensors, 360° sensors, 360° sonar sensors, and any form of proximity sensors in the invention to follow a thermoplastic paint for blind individuals and avoid obstructions.
